(19) United States
(12) Patent Application Publication
(10) Pub. No.: US 2013/0210717 A1
(43) Pub. Date: Aug. 15, 2013

(54) GENE THERAPY FOR DIABETES WITH CHITOSAN-DELIVERED PLASMID ENCODING GLUCAGON-LIKE PEPTIDE 1

(71) Applicants: Abderrazzak Merzouki, Laval (CA); Michael D. Buschmann, Montreal (CA)

(72) Inventors: Abderrazzak Merzouki, Laval (CA); Michael D. Buschmann, Montreal (CA)

(73) Assignee: CORPORATION DE L'ECOLE POLYTECHNIQUE DE MONTREAL, Montreal, QC (CA)

(21) Appl. No.: 13/697,082

(22) PCT Filed: May 10, 2011
(86) PCT No.: PCT/CA2011/000546

§ 371 Date: Jan. 21, 2013

Related U.S. Application Data
(60) Provisional application No. 61/332,834, filed on May 10, 2010.

(51) Int. Cl. A61K 48/00; A61K 31/713; A61K 38/28

(52) U.S. Cl. CPC A61K 48/005; A61K 38/28; A61K 31/713
USPC 514/6.5; 514/44 R; 514/44 A

(57) ABSTRACT

Chitosan delivers a plasmid encoding Glucagon-Like Peptide 1 (GLP-1) to cells in a patient for gene therapy of diabetes. Chitosan is optimized for plasmid transfection by modulating three of its physico-chemical properties: degree of deacetylation (DDA), molecular weight (MW), and ratio of amines on chitosan to phosphates on DNA (N:P ratio), Chitosan 92-10-5 (DDA-MW-N:P) is more efficient than chitosans 80-10-10 and 80-80-5 in delivering a plasmid encoding luciferase or GLP-1(7-37) to cells. In the Zucker Diabetic Fatty (ZDF) rat model of diabetes, chitosan-delivered pVax plasmid encoding GLP-1 lowers glucose levels, increases insulin production and reduces weight gain.

### CROSS-REFERENCE TO RELATED APPLICATIONS

The present application claims priority on U.S. Provisional Application No. 61/332,834, filed May 10, 2010, and incorporated herein by reference.

### TECHNICAL FIELD

The invention relates to an improved composition and method for the efficient non-viral delivery of nucleic acids to cells using chitosan in order to treat type II diabetes mellitus related pathologies.

### BACKGROUND OF THE INVENTION

Glucose functions as a precursor for the synthesis of glycoproteins, triglycerides and glycogen. It also provides an important energy source by generating ATP through glycolysis. Glucose is a monosaccharide found either as a free molecule or derived from the catabolism of disaccharide or complex sugar chains. It is obtained directly from diet, primarily following the hydrolysis of ingested disaccharides and polysaccharides or by synthesis from other substrates in organs such as liver. Glucose derived from diet is transferred from the lumen of the small intestine to the blood. Both dietary glucose and glucose synthesized within the body have to be transported from the circulation into target cells. These processes involve the transfer of glucose across plasma membranes and occur via membrane transport proteins. At the level of the small intestine, glucose is transported via an energy-dependent Na+/glucose co-transporter in order to achieve its efficient absorption. In the kidney, the filtered glucose is reabsorbed into the blood.

In contrast to the highly specific tissue expression of the Na+/glucose dependant transporters, all mammalian cells contain one or more members of the facilitative glucose transporter family (GLUT). These transporters are characterized by a stereo selectivity allowing the bidirectional transport of glucose between the extracellular and intracellular spaces within the body and thereby assuring a constant supply of circulating glucose available for metabolism.

Metabolic disorders lead to a variety of diseases. Type II diabetes mellitus is one such metabolic disorder that affects glucose homeostasis and accounts for 90% of all diabetes worldwide (Wild et al., 2004, Diabetes Care, 27: 1047-1053). According to the Canadian Diabetes Association, more than two million Canadians have diabetes while a U.S. study indicates that diabetes costs the Canadian healthcare system $13.2 billion per year with costs rising rapidly (Dawson et al., 2002, Diabetes Care, 25: 1303-1307).

The aetiology and pathogenesis of type II diabetes (T2D) are multifactorial and heterogeneous. T2D leads to a disease with relative rather than absolute insulin deficiency due to the pancreatic β-cells which become progressively less able to secrete sufficient insulin to maintain the normal carbohydrate and lipid homeostasis (Bell and Polonsky, 2001, Nature, 414: 788-791). Metabolic abnormalities associated with T2D are caused in part by inadequate insulin action and result in or cause changes in the gene expression in the skeletal muscle. Recently, T2D has been linked to mutations in homeodomain transcription factor IDX-1 that plays a role in β-cell development and insulin activation (Habener, 2002, Drug News Perspect, 15: 491-497).

Glucose metabolism is regulated by a number of peptide hormones, including insulin, insulin like growth factor (IGF), glucagon and incretins. The complex mechanism by which these peptide hormones regulate this metabolism and how they affect each other is partially elucidated. For example, glucagon stimulates the release of stored glucose and thus raising blood levels as well as the secretion of insulin, a glucose intake promoting peptide, in order to maintain homeostasis. Glucagon binds to receptors on the surface of pancreatic β-cells which produce insulin and in consequence promote its secretion. Incretins are gut derived hormones that stimulate insulin postprandial secretion in response to food consumption before blood glucose levels rise. This phenomenon is known as the incretin effect. Glucagon like peptide-1 (GLP-1) is an incretin hormone that promotes glucagon inhibition, insulin expression and secretion. It has a tropic effect on β-cells and prevents their apoptosis thus lowering postprandial glucose level, in a glucose dependant manner, avoiding hyperglycemia.

GLP-1 originates from enzymatic processing of the glucagon precursor, pro-glucagon, a 180 amino acid peptide. This transformation is catalyzed by protein convertase PC1/3 to yield tGLP-1, which is subsequently transformed into the active GLP-1. GLP-1 is a potential therapeutic agent for type II diabetic patients and is now a focus of the pharmaceutical industry.

Multiple mammalian studies, including human, have demonstrated insulinotropic responses to exogenous administration of GLP-1, particularly GLP-1 (7-36) NH₂ and GLP-1 (7-37). For example, a 6-week subcutaneous infusion of GLP-1 in patients with type II diabetes, achieving plasma levels of GLP-1 in the 60-70 pmol/L range, produced substantial improvements in insulin secretory capacity and insulin sensitivity (a reduction in HbA1c of 1-2%, and a modest weight loss) (Zander et al., 2002, Lancet, 359: 824-830). However, it has been demonstrated that the half life of GLP-1 is very short and that less than 10% of the administrated GLP-1 is intact and biologically active only a few minutes after injection. This is mainly due to the action of the dipeptidyl peptidase IV (DPP-IV) enzymes that cleave the His:Ala:Glu sequence at the N-terminal region of the GLP-1 (Hansen et al., 1999, Endocrinology, 140: 5356-5363).

Therapeutic approaches for enhancing incretin action include degradation-resistant GLP-1 receptor agonists and inhibitors of dipeptidylpeptidase-IV (DPP-IV) activity, a class of drugs known as incretin enhancers. For example, the incretin mimetic GLP-1 agonist exenatide 4 (half life of 60 to 90 minutes) discovered in lizard venom showed reductions in fasting and postprandial glucose concentrations, plasma HbA1c (glycated hemoglobin related to plasma glucose concentration) and mild weight loss in phase III clinical trials (De Fronzo et al., 2005, Diabetes Care, 28: 1092-1100). Additionally, the GLP-1 receptor agonist liraglutide (Victoza™) has been approved in Europe for the treatment of diabetes mellitus type II and represents a human GLP-1 analogue which is applied once a day. Moreover, orally administered DPP-iv inhibitors, such as Sitagliptin™ and Vildagliptin™, reduce HbA1c by 0.5-1.0%, with few adverse effects and no weight gain (Herman et al., 2005, Clin Pharmacol Ther, 78: 675-688).

However, given the very short half life of GLP-1 (3 to 5 min) due to the activity of dipeptidyl-peptidase IV (DPP-IV), the development of efficient targeted GLP-1 gene delivery systems for sustained expression to enhance glycemic control is required. The main disadvantage of these GLP-1 analogs is that they require repeated administration by subcutaneous injection. An alternative means of sustaining GLP-1 activity is by gene delivery to host cells to extend the synthesis of the peptide in an active form. This can be achieved through delivery of plasmid encoding GLP-1 using vectors for gene therapy. DNA based strategies to maintain expression of GLP-1 peptide have been successful in a number of animal studies. For example, a fusion gene encoding the active human GLP-1 and mouse IgG1 heavy-chain constant regions (GLP-1-Fc) were generated (Soltani et al., 2007, Gene Ther, 14: 981-988) and injected into T2D db/db mice without any delivery vector. The results demonstrated that the expression of GLP-1/Fc peptide normalized glucose tolerance by enhancing insulin secretion and suppressing glucagon release. The therapeutic effects were observed several months after administration of the DNA construct. Furthermore, adenoviral gene delivery of a GLP-1 modified vector into Balb/c and db/db mice, ob/ob mice and ZDF rats showed similar results (Lee et al., 2007, Diabetes, 56: 1671-1679). For example Lee et al. (2007, Diabetes, 56: 1671-1679), showed that circulating GLP-1 was significantly increased in ob/ob rAd-GLP-1 (recombinant adenoviral GLP-1 expressing vector) treated mice for at least 4 weeks compared with rAd-βgal-treated diabetic and untreated normal mice, indicating that a substantial amount of circulating GLP-1 is exogenously produced by rAd-GLP-1 therapy. Their results restored normal glucose level by enhancing β-cell mass, insulin secretion, improvement of glucose uptake in adipocytes and suppression of glucagon release.

Although most ongoing gene therapy protocols rely on viral vectors, non-viral gene transfer is attracting increasing interest due to safety and low manufacturing cost advantages (Niidome et al., 2000, Biomaterials, 21: 1811-1819). One approach to non-viral gene delivery is to use cationic polymers that complex to plasmid DNA by electrostatic attraction forces to form nanoparticles, or therapeutic nanoparticles, which protect the plasmid from nuclease activity that can degrade DNA in seconds (Dash et al., 1999, Gene Ther, 6: 643-650). The main disadvantage of non-viral gene delivery has been low transgene expression levels compared to viral vectors. However, recent advances in nanoscience has achieved a tremendous improvement in transfection efficiencies and a lowered toxicity of such non-viral vectors for gene delivery.

Calcium phosphate is one example of known non-viral gene transfer methodology. However, a major drawback of this vector is its limited efficiency and its inability to protect nucleic acids from nuclease degradation. Despite the improvement of calcium phosphate's ability to protect nucleic acids, its transfection efficiency did not improve thus preventing its effective use in vivo.

Cationic lipids form complexes with nucleic acids via electrostatic interaction eventually forming multi lamellar lipid-nucleic acid complexes (lipoplexes). The liposome formulations usually include a cationic lipid and a neutral lipid such as DOPE (dioleoylphosphatidylethanolamine). The neutral lipid contributes to the stability of the liposomic formulation and facilitates membrane fusion. In addition, it contributes to the lysosomal escape by destabilizing the endosome. Lipoplexes are one of the most efficient ways of delivering nucleic acid into cultured cells. Despite their transfection efficiency, lipoplexes are toxic as observed in cultured cells and confirmed by several in vivo findings. The toxicity is closely associated with the charge ratio of the cationic lipids to the nucleic acid in the complex as well as the administered dose. More biocompatible formulations are being tested and developed in order to reduce lipoplexe associated toxicity. Reduction of toxicity is mainly achieved via grafting with other cationic polymers or by reducing the total charge of the polymer.

Cationic polymers form polyplexes of nanometric size by a strong interaction between oppositely charged polycation and nucleic acids. These polyplexes encapsulate nucleic acids thus preventing their degradation by nuclease activity (Romoren et al., 2003, Int J Pharm, 261: 115-127). A large number of natural and synthetic cationic polymers have been used as vehicle for gene delivery or silencing. Many of these polyplexes that use cationic polymers have superior transfection efficiency and lower serum sensitivity compared to lipoplexes. The group of synthetic polycations includes peptides such as poly-L-Lysine (PLL) and poly-L-ornithine as well as polyamines such as polyethylenimine (PEI), polypropylenimine, and polyamidoamine dendimers (PAMAM).

An advantage of polyplexes is that their formation does not require interaction of multiple polycations; on the contrary, liposomes need multiple lipid components which make their macroscopic properties easier to control. Another major advantage of polycations is their block structures which allow direct chemical modification to attain higher efficiency or specific cell targeting. However, despite these advantages, many cationic polymers have been found toxic because of their surface charge density since high charge density polyplexes appear to be more toxic. Furthermore, it has been reported that the charge density in the polymer plays a more important role in cytotoxicity than the total amount of charge. Toxicity may be molecular weight dependent and the cytotoxicity of PEI increases linearly with its molecular weight. Moreover, accumulation of non degradable polymer such as PEI in the lysosome, a phenomenon called lysosomal loading, may yet be an additional contributor to toxicity.

GLP-1 encoding plasmids have been delivered both in vivo and in vitro using the polymeric agent poly [α-(4-aminobutyl)-L-glycolic acid] (PAGA) for the purpose of developing a method and a composition for the treatment of type II diabetes (US patent application publication No. 2003/0220274). In their published patent application, Oh and collaborators claim that GLP-1 expression under the control of a chicken β-actin promoter resulted in normalized blood glucose levels. A major drawback with their construct is the lack of control on the insert expression using the chicken β-actin promoter.

It would be highly desirable to be provided with efficient targeted GLP-1 gene delivery systems for sustained expression to enhance glycemic control. It would thus be highly desirable to be provided with an improved composition and methodology to increase the delivery of GLP-1 encoding plasmids for the treatment of type II diabetes.

### SUMMARY OF THE INVENTION

One aim of the present invention is to provide a composition comprising chitosan and a plasmid DNA sequence encoding for Glucagon like peptide-1 (GLP-1), a GLP-1 variant or a GLP-1 derivative.

It is also provided a composition as defined herein for the treatment of diabetes mellitus or related conditions in a patient.

It is also provided the use of the composition as defined herein for the treatment of diabetes mellitus or related conditions in a patient, for the control of glucose metabolism in a patient, and for the treatment of a metabolic disease in a patient.

In other embodiments, it is provided the use of a composition as defined herein in the manufacture of a medicament, biologic or drug for the treatment of diabetes mellitus or related conditions in a patient, for the control of glucose metabolism in a patient, and for the treatment of a metabolic disease in a patient.

In other embodiments, it is provided a method for treating diabetes mellitus or related conditions in a patient; a method for the control of glucose metabolism in a patient; and a method for treating metabolic disease in a patient comprising administering to the patient an effective amount of the composition as defined herein.

In an embodiment, the GLP-1 variant is GLP-1(7-34), GLP-1(7-35), GLP-1(7-36), Val⁸-GLP-1(7-37), Gln⁹-GLP-1(7-37), D-Gln⁹-GLP-1(7-37), Thr¹⁶-Lys¹⁸-GLP-1(7-37), Lys¹⁸-GLP-1(7-37), His⁷-GLP-1 (7-37), Ser⁸-GLP-1(7-37) or Tyr⁹-GLP-1(7-37).

In another embodiment, the GLP-1 variant is SEQ ID NO:3 or SEQ ID NO:4.

In a further embodiment, the chitosan is heterogeneously deacetylated.

In another embodiment, the plasmid DNA is a safe plasmid for genetic immunization.

In an additional embodiment, the plasmid DNA comprises an expression facilitating sequence derived from a CMV promoter (CMV Pro); a sequence coding for a furin cleavage site (FCS); and a sequence coding for GLP-1, GLP-1 variant or GLP-1 derivative thereof that is operably linked to the expression facilitating sequence.

In a particular embodiment, the plasmid DNA is pVax1 plasmid.

In a further embodiment, the chitosan has a molecular weight of 7 kDa to 150 kDa and a deacetylation degree (DDA) of 75% to 95%, particularly the chitosan is 5 to 15 kDa and the DDA is 90% to 95%.

In a further embodiment, the ratio of amine groups on chitosan to phosphate groups of plasmid DNA (N:P ratio) is in the range of 2 to 20, particularly the N:P ratio is of 3 to 10.

In another embodiment, the chitosan comprises block distribution of acetyl groups or a chemical modification.

In a further embodiment, diabetes mellitus related conditions are insulin-dependent diabetes mellitus (type I diabetes), noninsulin-dependent diabetes mellitus (type II diabetes), insulin resistance, hyperinsulinemia, diabetes-induced hypertension, obesity, damage to blood vessels, damage to eyes, damage to kidneys, damage to nerves, damage to autonomic nervous system, damage to skin, damage to connective tissue, and damage to immune system.

In an embodiment, the composition described herein controls the glucose metabolism in a patient, reduces the blood glucose level in the patient and is for the treatment of a metabolic disease in a patient.

In a further embodiment, the composition reduces the weight gain in the patient.

In another embodiment, the composition reduces circulating half life of incretins, incretin-like proteins, or glycoregulating proteins, and increases insulin secretion and β-cells proliferation.

In an additional embodiment, the composition is formulated for a subcutaneous administration, an intramuscular administration, an intravenous administration, an intradermal administration, intramammary administration, an intraperitoneal administration, an oral administration or a gastrointestinal administration.

In an additional embodiment, the composition also comprises insulin or a hypoglycemic compound, such as metformin, acarbose, acetohexamide, glimepiride, tolazamide, glipizide, glyburide, tolbutamide, chlorpropamide, thiazolidinediones, alpha glucosidase inhibitors, biguanindine derivatives, troglitazone, or a mixture thereof.

In an additional embodiment, the composition is formulated for concurrent administration with a small interference RNA's (siRNAs), a suitable delivery reagent, insulin or a hypoglycemic compound. The delivery agent can be Mirus Transit TKO® lipophilic reagent, Lipofectin®, Lipofectamine™ Cellfectin®, polycations or liposomes.

In another embodiment, the patient is an animal or a human.

The expression “metabolic disorder(s) or disease(s)” enclosed herewith is intended to encompass any medical condition characterized by problems with an organism's metabolism, such as central obesity, hypertension, wasting syndrome (cachexia), atherogenic dyslipidemia, and chronic inflammation associated with metabolic syndrome.

### BRIEF DESCRIPTION OF THE DRAWINGS

Reference will now be made to the accompanying drawings.

FIG. 1 illustrates native GLP-1 and variants of GLP-1 constructs.

FIG. 2 illustrates the nucleic acid sequence of the proglucagon encoding gene, wherein the glucagon, glucagon like peptide-1 and -2 are shown, with highlighted sequences corresponding to start codon, stop codon and to polyadenylation sites, and boxes correspond to sequences used for primer generation.

FIG. 3 illustrates luciferase reporter gene bearing chitosan nanoparticles used in vitro to transfect cells, wherein chitosan 92-10-5, 80-10-10 and 80-80-5 were used to transfect (a) HepG-2, (b) Caco-2 and (c) HT-29 cell lines, with positive (Lipofectamine™) and negative (untreated cells) controls.

FIG. 4 illustrates the ability of chitosan-based formulations to protect recombinant plasmids from DNAse I digestion, wherein pVax1-GLP-1 Chitosan-based formulations (92-10-5, 80-10-10 and 80-80-5) were incubated in presence of different DNAse I concentrations and in A) nucleic acid bands correspond to the recombinant plasmids compared with controls (C1: pVax1-GLP-1 incubated without DNAse I and chitosanase; C2:pVax1-GLP-1 incubated without DNAse I and with chitosanase; and C3: pVax1-GLP-1 incubated with 0.5 unit of DNAse I and chitosanase); and wherein in B) the relative amount of pVax1-GLP-1(%) determined by comparison of the treated sample intensity versus the non-treated sample intensity (0 Unit of DNAse=100% of intensity) is shown.

FIG. 5 illustrate the cellular uptake of pVax1-GLP-1/92-10-5 nanoparticles in HepG-2 cell line, images obtained by confocal microscopy using rhodamine labeled chitosan and FITC labeled pVax-GLP-1 plasmid, images were taken 4 hours (a) and 24 hours (b) post transfection (panels 1: FITC detection to localize the recombinant plasmid pVax1-GLP-1 (green); 2: rhodamine detection to localize chitosan (red); 3: cells without any detection; and 4: detection of both FITC and rhodamine (yellow)).

FIG. 6 illustrates expression levels of GLP-1 (7-37) and its variant forms in HepG-2 transfected cells using different chitosan-based formulations (values are expressed as mean±s.d.; n=3 rats/group. *p<0.05, ** p<0.01 compared with pVax-GLP-1 alone (no chitosan); statistical analyses used the General Linear Model and Contrast Analyses with Treatment as predictor).

FIG. 7 illustrates the quantification of GLP-1 (7-37) expression in ZDF rat model using different chitosan-based formulations (values are expressed as mean±s.d.; n=3 rats/group. *p<0.05, ** p<0.01 compared with pVax-GLP-1 alone (no chitosan); statistical analyses used the General Linear Model and Contrast Analyses for each Day with Treatment as categorical predictor).

FIG. 8 illustrates glucose tolerance test results in ZDF rats after completion of the chitosan/pVax1-GLP-1 injection schedule. Glucose tolerance was measured at 0.5, 1, 2 and 3 hours following the glucose injection. AUC corresponds to the area under the curve. Glucose concentration was measured directly on blood samples using photometry techniques (values are expressed as mean±s.d.; n=3 rats/group. *p<0.05, ** p<0.01 compared with pVax-GLP-1 alone; statistical analyses used the General Linear Model and Contrast Analyses with Treatment as predictor).

FIG. 9 illustrates the evaluation of insulin production in ZDF treated rats with different chitosan/pVax1-GLP-1 formulations. *Statistical analyses using the General Linear Model indicated that treatment (Intramuscular or IM and subcutaneous or SC) had a significant effect on insulin concentration (*p<0.05).

FIG. 10 illustrates efficacy and longevity of therapeutic effect of chitosan-based nanoparticles measured by intraperitonial glucose tolerance test. Glucose values are peak values at 60 minutes expressed as means±s.d.; n=3 rats/group. *p<0.05, ** p<0.01 compared with pVax-GLP-1 alone (no chitosan). Statistical analyses used the General Linear Model and Contrast Analyses for each Day with Treatment as categorical predictor.

FIG. 11 illustrates the effect of recombinant GLP-1 (in different chitosan-based formulations) on weight of treated ZDF rats versus untreated ZDF rats (values are expressed as means±s.d.; n=3 rats/group. *p<0.05, compared with pVax-GLP-1 alone; statistical analyses used the General Linear Model and Contrast Analyses for each Day with Treatment as categorical predictor).

FIG. 12 illustrates the histological examination of muscle and skin (safranin-O/fast-green/iron-hematoxylin) following chitosan/pVax-GLP-1 nanoparticles administration; (a) and (b) are tissue from the IM injection sites sampled 1 day following administration of chitosan-based formulations; (c) and (d) are tissue from the IM injection sites sampled 3 days following administration; (e) and (f) are tissue from the IM injection sites sampled 14 days following administration; (g) and (h) are tissue from the SC injection sites sampled 3 days following administration.

FIG. 13 illustrates environmental scanning electron microscope (ESEM) images showing spherical shape of pVax1-GLP-1/92-10-5 nanoparticles ((a) pVax-GLP-1/92-10-5; (b) pVax-GLP-1/80-10-10; and (c) pVax-GLP-1/80-80-5).

### DETAILED DESCRIPTION

In accordance with the present description, there is provided a composition and method for non-viral delivery of nucleic acids to cells and organs in order to treat type II diabetes mellitus related pathologies.

The present description provides methods for treatment of diabetes mellitus and related conditions and symptoms. Such diabetes mellitus and related conditions include insulin-dependent diabetes mellitus (type I diabetes), noninsulin-dependent diabetes mellitus (type II diabetes), insulin resistance, hyperinsulinemia, and diabetes-induced hypertension. Other diabetes-related conditions include obesity and damage to blood vessels, eyes, kidneys, nerves, autonomic nervous system, skin, connective tissue, and immune system. The composition described herein can be used either alone or in combination with insulin and/or hypoglycemic compounds.

As used herein, “treatment” and “treating” include preventing, inhibiting, and alleviating diabetes mellitus and related conditions and symptoms. The treatment may be carried out by administering a therapeutically effective amount of the composition described herein. In other instances, the treatment may be carried out by concurrently administering a therapeutically effective amount of a combination of insulin and the composition described herein. In still other instances, the treatment may involve concurrently administering a therapeutically effective amount of a combination of a hypoglycemic compound and the composition described herein when the diabetes mellitus and related conditions to be treated is type II diabetes, insulin resistance, hyperinsulinemia, diabetes-induced hypertension, obesity, or damage to blood vessels, eyes, kidneys, nerves, autonomic nervous system, skin, connective tissue, or immune system.

The composition disclosed comprises a non viral vector for the efficient delivery of nucleic acid entities such as DNA vectors to cells, tissues and organs in mammals, e.g., human. In particular, it is described specific chitosan compositions to ensure high expression of GLP-1 protein for therapeutic use in type II diabetes. The disclosed composition is the first non-viral sustained release therapeutic gene delivery system shown to increase circulating GLP-1 to therapeutic levels in a type II diabetes animal model.

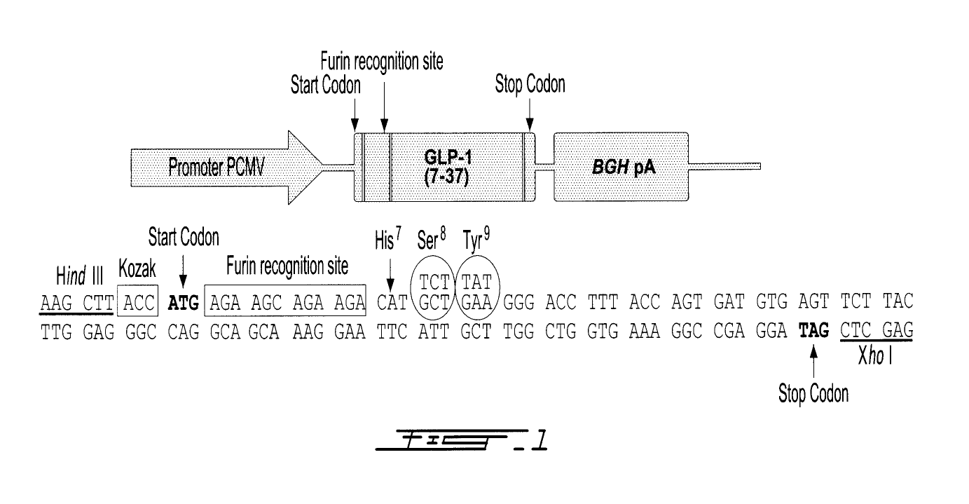

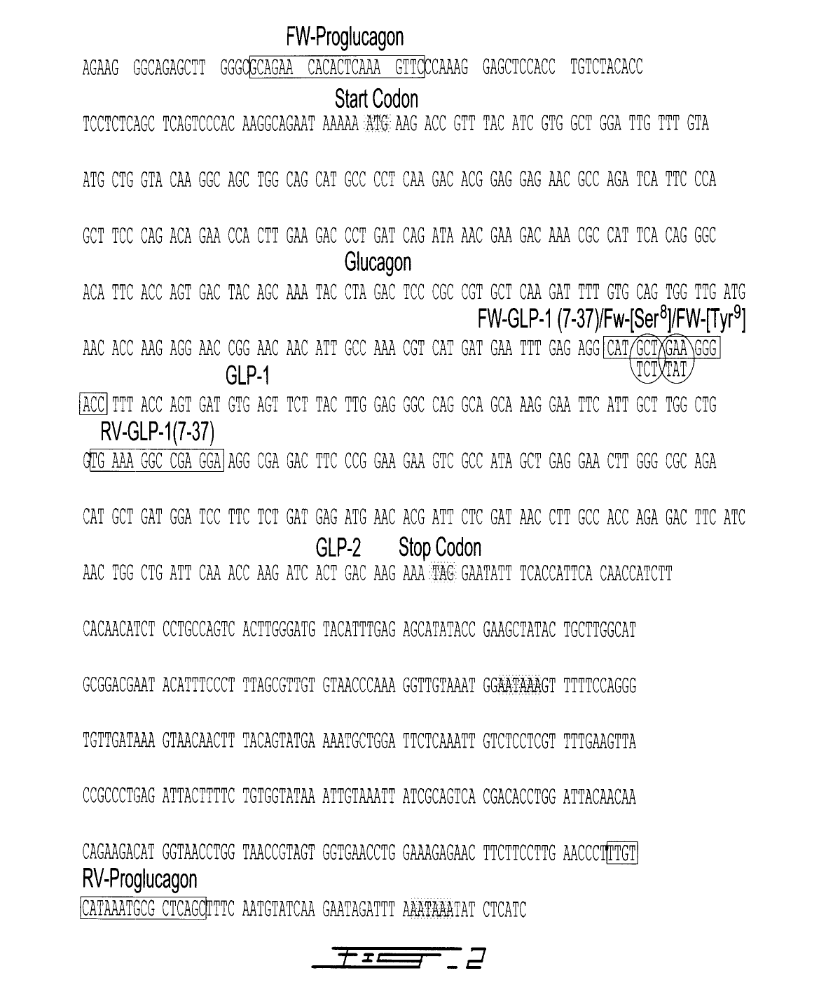

A therapeutic GLP-1 coding DNA plasmid using the eukaryotic recombinant expression vector pVax1 was produced (see FIGS. 1 and 2). The vector disclosed herein is a highly safe plasmid for genetic immunisation in animals since all plasmid elements have been optimized to comply with FDA guidelines for design of DNA vaccines regarding content and elimination of extraneous materials. The eukaryotic DNA sequences in the plasmid are limited to those required for expression to minimize the possibility of chromosomal integration and a kanamycin resistance gene for selection in E. coli minimizes allergic responses in hosts.

Furthermore, the composition described herein can be used in order to provide symptomatic relief, by administering GLP-1 inducing entities to a subject at risk of or suffering from type II diabetes within an appropriate time window prior to, during, or after the onset of symptoms.

A composition comprising chitosan/pVax1 plasmid DNA has a great potential as a gene carrier for recombinant protein expression. Intramuscular (IM) and subcutaneous (SC) administration of a chitosan/pVax1 plasmid DNA led to the expression and distribution of FGF-2 and PDGF-BB recombinant proteins in surrounding tissues, and eventually in serum (Jean et al., 2009, Gene Ther, 16: 1097-1110). The recombinant proteins were still detectable at the injection site and surrounding tissues several weeks post administration. This implies that the chitosan/plasmid DNA nanoparticles were effectively captured by tissues and cells rather than being broken down rapidly.

It is described herein several GLP-1 variants, and their increased biopersistance due to their resistance to DPP-IV degradation is also disclosed. The pVax1 plasmid described herein is an FDA approved vector for vaccine development when compared to other vectors, such as the pBeta vector described in U.S. patent application publication No. 2003/0220274, which is a mammalian expression vector.

One advantage of the composition described herein is that, compared to the composition disclosed for example in U.S. patent application publication No. 2003/0220274 wherein the in vitro transfection of HepG-2 using 2 μg or 4 μg of pBeta-GLP-1(7-37) plasmid yielded a concentration of 8.3 ng/L and 20 ng/L GLP-1, transformation of HepG-2 using 2.5 μg pVax-GLP-1 (7-37) carried by a specific chitosan formulation yielded a significantly higher GLP-1 concentration (30 ng/L). This result is not surprising in light of the results described herein below which show gene expression levels to be very much dependent on specific polymer characteristics. This is probably due to a higher expression rate and a more efficient delivery of chitosan formulation versus the PAGA polymer. In addition, several reports in the art show that PAGA particle size range from 250-500 nm with an average size of 350 nm (Lim et al., 2000, Pharm Res, 17: 811-816) which lack the demonstrated non-toxicity of chitosan vectors. The results disclosed herein show smaller particle size ranging between 150-250 nm which make them more efficient from a biodistribution standpoint (in vivo transfection efficiency) and increase their circulating half life by efficient renal clearance circumvention.

It is demonstrated herein that the compositions described herein are effective gene expression vectors achieving transfection efficiencies similar to the commercial liposome Lipofectamine™. Moreover, the composition achieved comparable result in delivering nucleic acid into cells and similar expression results as Lipofectamine™.

Chitosan is a natural polymer of glucosamine and N-acetyl-glucosamine monomers linked by β-1, 4 glycosidic bonds and is derived from alkaline deacetylation of chitin. The molecular weight and the degree of deacetylation (DDA) of chitosan dictate its biological and physicochemical properties. The degree of deactylation of chitosan is the percentage of glucosamine monomers (100% DDA is polyglucosamine while 80% DDA has 80% glucosamine and 20% N-acetyl-glucosamine). For example, chitosan biodegradability is affected by the amount and the distribution of acetyl groups. The absence of these groups or their random, rather than block, distribution results in very low rate of degradation.

Chitosan possesses a wide range of beneficial properties including biocompatibility, biodegradability, mucoadhesive properties, antimicrobial/antifungal activity, and very low toxicity.

Many studies have addressed the effect of chitosan molecular weight (MW) and degree of deacetylation (DDA) on nanoparticle uptake, nanoparticle trafficking, and transfection efficiency on different cell lines. Huang et al. (2005, Journal of Controlled Release, 106: 391-406) addressed this subject on A549 cells. However, this study only used 7 formulations (chitosan of 10, 17, 48, 98 and 213 kDa at 88% DDA; 213 kDa at 61 and 46% DDA) to study the effect of MW and DDA on transfection efficiency. They found that a decrease in MW and DDA renders lower transfection efficiency. However, the relationship between these two parameters is much more complex and demands accounting for the effects of both of these two parameters to achieve optimal stability. Moreover, only one parameter at a time was varied preventing an appreciation of the coupling effect between MW and DDA and relation to the pH of the transfection media and to chitosan-DNA ratio. Another study addressing this complex relation has been achieved by Layertu et al. (Biomaterials, 27: 4815-4824). In their study, they varied the molecular weight and the DDA systematically and independently as well as the chitosan-DNA ration (N:P) and/or the pH of the transfection media. This comprehensive study demonstrated that optimal high transfection efficiencies comparable to the broadly used commercial liposome (Lipofectamine™) in HEK293 cells could be achieved with specific chitosans (U.S. patent application publication No. 2009/0075383).

The DNA binding capacity of chitosan increases when its degree of deacetylation increases due to a higher charge density along the chain. Thus chitosans with a DDA that is too low are unable to bind efficiently DNA and cannot form physically stable complexes to transfect cells. DDA also exerts a dominant influence on biodegradability where high DDAs are difficult to degrade. In this light, a recent study by Koping-Hoggard et al. (2001, Gene Ther, 8: 1108-1121) suggested that endosomal escape of the high MW chitosan based complexes depend on enzymatic degradation of chitosan that would occur less readily with high DDA chitosans. The resulting degradation fragments are hypothesized to increase endosome osmolarity and lead to membrane rupture. Thus, for highly deacetylated chitosan, reduced degradability could result in reduced endosomal escape.

The influence of chitosan MW on the ability to bind nucleic acids was evaluated in several studies. Binding affinity between oppositely charged macromolecules is electrostatically driven and therefore is strongly dependant on the valence of each molecule, with a low valence yielding only weak binding. The reduction in chitosan valence for lower MW with shorter chains has been shown to reduce its affinity to DNA (Koping-Hoggard et al., 2003, J Gene Med, 5:130-141). Although complex stability is desirable extracellularly, MacLaughlin et al. (1998, J Control Release, 56: 259-272) suggested that a high MW chitosan can form complexes that are too stable to transfect cells since they cannot be disassembled once inside the cell and thus remain inactive. Furthermore, Layertu et al. (2006, Biomaterials, 27: 4815-4824) and Ma et al. (2009, Biomacromolecules, 106: 1490-1499) showed that MW does not appear to be a dominant factor in cellular uptake but that MW appears to play a role in nucleic acid binding affinity where longer chains bind more tightly to DNA.

The amine (N) to phosphate (P) ratio has been found to play an important role in DNA binding. For example, increasing the N:P ratio enhances chitosan binding to DNA. For the same DDA, a lower MW chitosan requires a higher N:P ratio to completely bind DNA. Similarly at equal MW, a lower DDA requires a higher N:P ratio to completely bind DNA (Koping-Hoggard et al., 2001, Gene Ther, 8: 1108-1121).

pH has been shown to play an important role in transfection efficiency. Layertu et al. (2006, Biomaterials, 27: 4815-4824) showed that complexes are more stable and an increase in transfection efficiency is achieved in slightly acidic medium. This can be explained by the fact that pH reduction increases chitosan protonation and thereby the positive its binding affinity to DNA as well as to negatively charged cell surface molecules to promote cell uptake.

The combined effect of the chitosan formulation parameters (DDA, MW, N:P and pH) was studied by Layertu et al. (2006, Biomaterials, 27: 4815-4824). Interestingly, they found that maximum transgene expression occurs for DDA: MW values that run along a diagonal from high DDA/low MW to low DDA/high MW. Thus if one increases/decreases DDA, one must correspondingly decrease/increase MW to maintain maximal transfection. As mentioned above, pH plays an important role in transfection efficiency since an increase in pH displaces the MW for the most efficient formulation toward higher MW because of the destabilization effect of pH by reducing chitosan protonation. On the other hand, for a given DDA, a change in N:P ratio from 5:1 to 10:1 displaces the MW for the most efficient formulation towards lower MW, probably because of the stabilizing effect of increasing chitosan concentration. Thus one can see the importance of these different formulation parameters on transfection efficiency and in the development of a more efficient and stable chitosan formulation.

Chitosan was used to deliver pharmacologically active compounds through different administrational routes including intranasal, oral, intra-peritoneal, and intramuscular routes. Chitosan/insulin was administered through intranasal routes in rat and sheep. These formulation involved the use of a water soluble chitosan (U.S. Pat. No. 5,554,388) of molecular weight of 10 kDa or greater, with no specification on degree of deacetylation.

Chitosan has also been used as adjuvant for the immunization of mice through an intranasal route with soluble formulations (Ilium and Chatfield, Vaccine composition including chitosan for intranasal administration and use thereof, 2002, West Pharmaceutical Services Drug Delivery & Clinical Research Centre Limited). These formulations involved chitosan glutamate with a MW ranging between 10-500 kDa with a degree of deacetylation between 50-90%.

Chitosan has also been used to deliver a variety of nucleic acids varying from plasmid DNA, to siRNA in vitro and in vivo as well. For example, chitosan/siRNA nanoparticles mediated TNF-α knockdown in peritoneal macrophages for anti-inflammatory treatment in an arthritis murine model (Howard et al., 2009, Mol Ther, 17: 162-168).

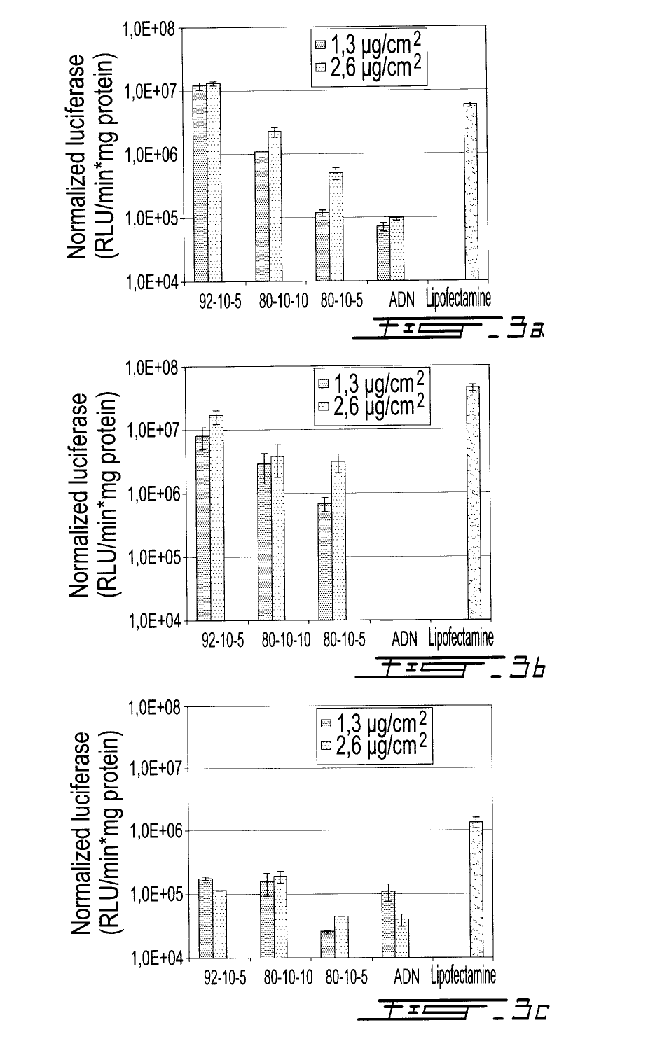

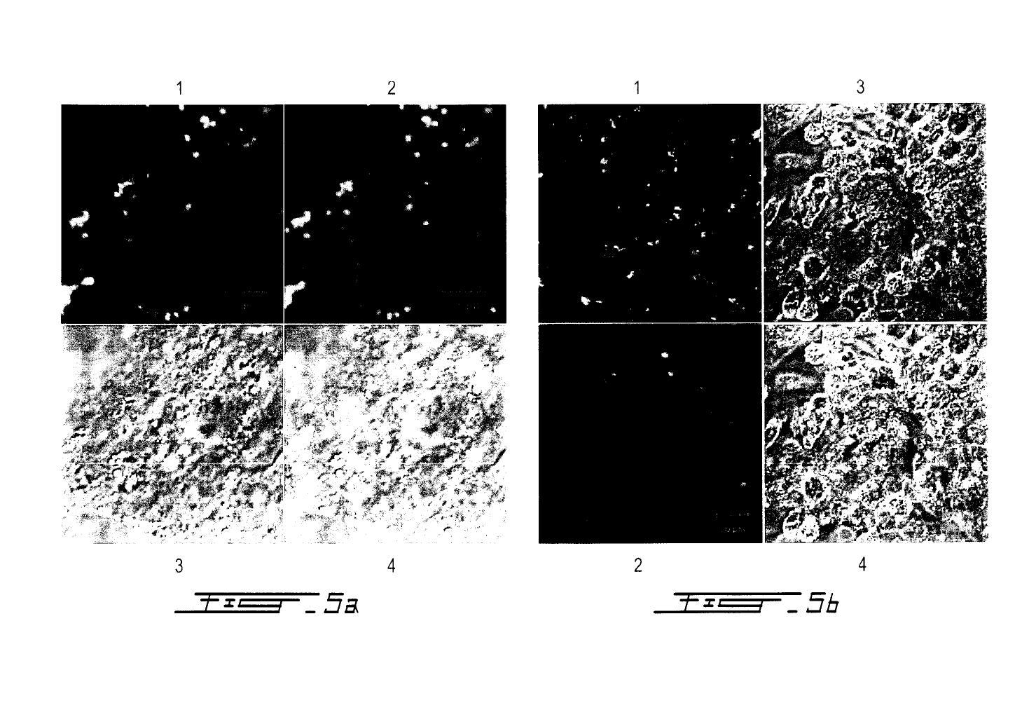

The chitosan formulation 92-10-5 showed highest transfection efficiency in Caco-2, HT-29 and HepG2 cell lines and was similar to commercial phospholipid systems (Lipofectamine™ in FIG. 3). These results revealed the potential of chitosan/plasmid-DNA systems as GLP-1 (and GLP-1 analogs) therapeutic delivery systems (see FIGS. 5 & 6).

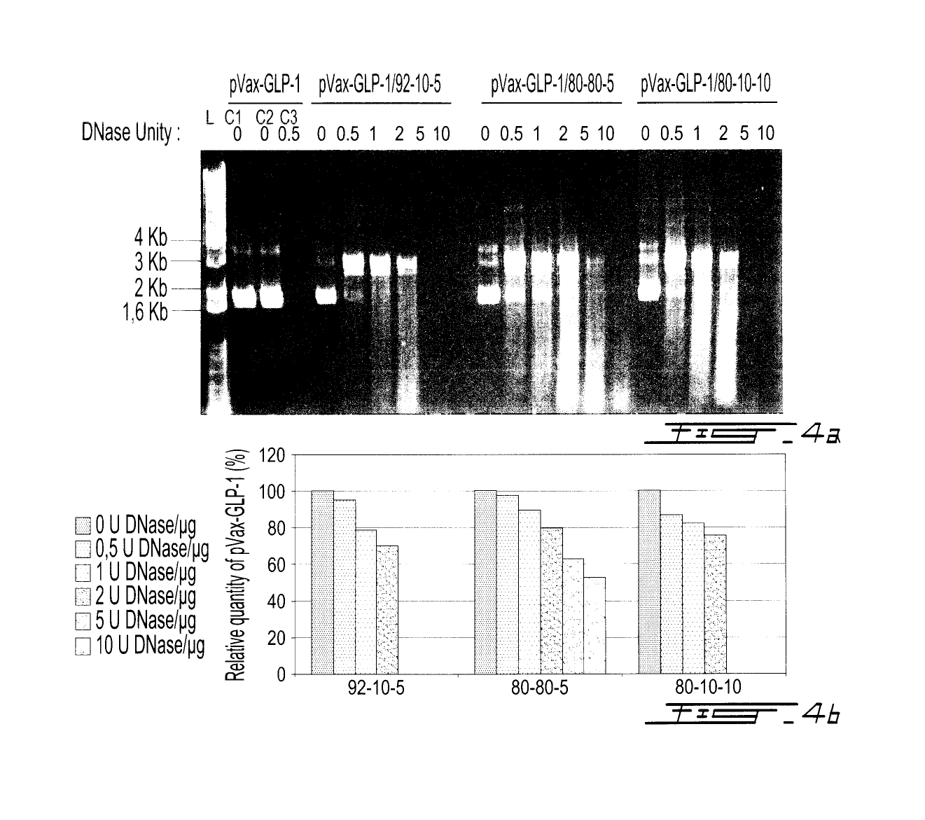

It is demonstrated herein the ability of the formulations to protect plasmid DNA (FIG. 4). The protection is considerable and accounts for approximately 70% of complexes when using 2 units of DNAse I/μg of DNA whereas the negative control is completely digested when 0.5 units of DNAse I per μg of DNA is used. The protection remains efficient when increasing DNAse I concentration to 5 units per μg of DNA.

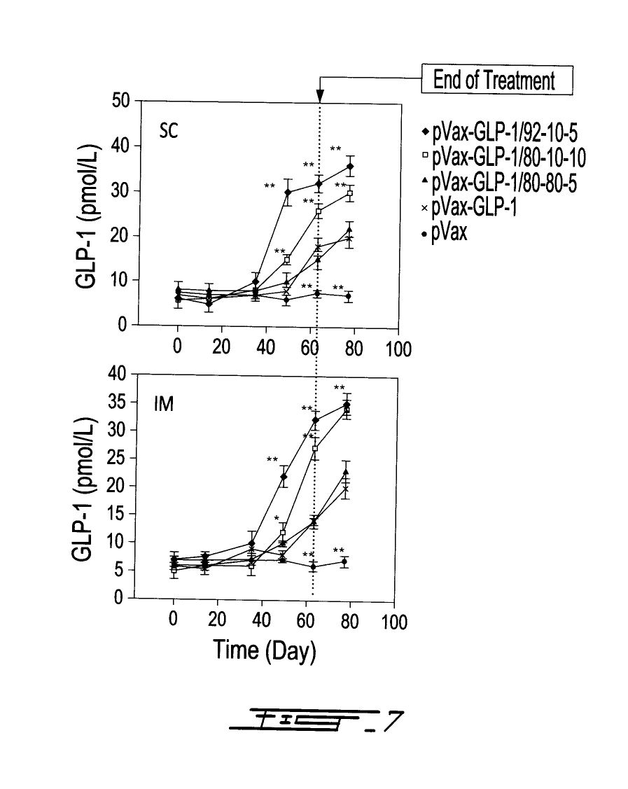

For in vivo studies, ZDF rats were used. After 49 days, a significant increase of active GLP-1 in the plasma of animals injected with nanoparticles was observed, the most efficient being 92-10-5 (FIG. 7).

The compositions and methods described herein may be applied for a variety of purposes, e.g, to deliver a variety of therapeutic proteins, to study the effect of different compounds on a cell or organism in the absence or reduced activity of the polypeptide encoded by the transcript, etc. Furthermore, the composition and methods may be applied in clinical therapy for type II diabetes and its related pathologies specifically to circumvent the short circulating half life of incretins and incretin-like proteins or any glycoregulating protein in order to treat diabetes (see FIGS. 7 to 11).

The composition contains a chitosan that has the following physicochemical properties: the combination of a number average molecular weight (Mn) such as in the range of 7 kDa to 80 kDa and a degree of deacetylation in the range of 80% to 95%. The chitosan molecule can also present block distribution of acetyl groups obtained by a heterogeneous treatment of chitin or can contain any chemical modification possible that increases transfection efficiency and maintain low toxicity or even lower it.

Examples of chitosan containing chemical modification are: chitosan-based compounds having: (i) specific or non-specific cell targeting moieties that can be covalently attached to chitin and/or chitosan, or ionically or hydrophobically adhered to a chitosan-based compound complexed with a nucleic acid or an oligonucleotide, and (ii) various derivatives or modifications of chitin and chitosan which serve to alter their physical, chemical, or physiological properties. Examples of such modified chitosan are chitosan-based compounds having specific or non-specific targeting ligands, membrane permeabilization agents, sub-cellular localization components, endosomolytic (lytic) agents, nuclear localization signals, colloidal stabilization agents, agents to promote long circulation half-lives in blood, and chemical derivatives such as salts, O-acetylated and N-acetylated derivatives. Some sites for chemical modification of chitosan include: C₂(NH—CO—CH₃ or NH₂), C₃(OH), or C₆(CH₂OH).

GLP-1 was cloned into pVax1 plasmid using a restriction enzyme based strategy. First, the GLP-1 sequence was amplified using a polymerase chain reaction on the proglucagon cDNA (FIG. 2) using a specific set of primers (RV-GLP-1(7-37), TCCTCGGCCTTTCT (SEQ ID NO:5); FW-GLP-1(7-37), CATGCTCAAGGGACC (SEQ ID NO:6); FW-[Ser⁸]GLP-1(7-37), CATTCTCAAGGGACC (SEQ ID NO:7); and FW4Tyr⁹1GLP-1(7-37), CATGCTTATGGGACC (SEQ ID NO:8)). In order to generate GLP-1 variants, the forward primer was modified to incorporate either Ser or Tyr at residues 8 and 9 respectively as shown in FIG. 2. Both native and variants sequences contain the His⁷ codon. The amplified products were cloned between Hind III and XhoI sites in the pVax1 plasmid (FIG. 1).

Encompassed herein is “GLP-1 analog” which is defined as a molecule having a modification including one or more amino acid substitutions, deletions, inversions, or additions when compared with GLP-1. GLP-1 analogs known in the art include, for example, GLP-1(7-34) and GLP-1(7-35), GLP-1(7-36), Val⁸-GLP-1(7-37), Gln⁹-GLP-1(7-37), D-Gln⁹-GLP-1(7-37), Thr¹⁶-Lys¹⁸-GLP-1(7-37), and Lys¹⁸-GLP-1(7-37), and such as disclosed in U.S. Pat. Nos. 5,118,666, 5,545,618 and 6,583,111. These compounds are the biologically processed forms of GLP-1 having insulinotropic properties.

Also encompassed is a “GLP-1 derivative”, defined as a molecule having the amino acid sequence of GLP-1 or of a GLP-1 analog, but additionally having at least one chemical modification of one or more of its amino acid side groups, a-carbon atoms, terminal amino group, or terminal carboxylic acid group. A chemical modification includes adding chemical moieties, creating new bonds, and removing chemical moieties. Modifications at amino acid side groups include acylation of lysine e-amino groups, N-alkylation of arginine, histidine, or lysine, alkylation of glutamic or aspartic carboxylic acid groups, and deamidation of glutamine or asparagine. Modifications of the terminal amino include the des-amino, N-lower alkyl, N-di-lower alkyl, and N-acyl modifications. Modifications of the terminal carboxy group include the amide, lower alkyl amide, dialkyl amide, and lower alkyl ester modifications. A lower alkyl is a C₁-C₄ alkyl. Furthermore, one or more side groups, or terminal groups, may be protected by protective groups known to the ordinarily-skilled protein chemist.

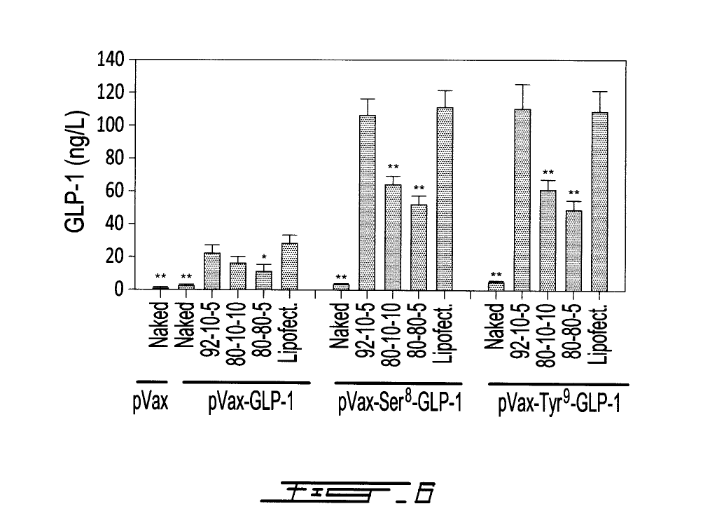

It is demonstrated herein that nanoparticles (pVax1-GLP1/92-10-5) were internalized into HepG-2 cells and plasmid (green) release was reached its maximum near 24 hours post transfection (FIG. 5). Moreover, GLP-1 expression reached approximately the same level when compared to the positive control (Lipofectamin™). Furthermore, GLP-1 variants with higher resistance to DPP-IV showed a fourfold higher expression level when compared with the native GLP-1 (FIG. 6).

The results on animals using the ZDF rat model showed promising results for the chitosan/plasmid-DNA system as a GLP-1 therapeutic delivery system in the treatment of type II diabetes mellitus (FIGS. 7 to 11).

After 49 days, a significant increase of active GLP-1 in the plasma of injected animals was observed, the most efficient being with the 92-10-5 composition (FIG. 7). Animals injected with nanoparticles (chitosan 92-10-5/[native-GLP-1(7-37)]) showed GLP-1 levels of about 5 fold higher (i.m injection) and 4 fold higher (s.c injection) than non-injected animals (FIG. 7) with a maximum concentration of active GLP-1 in the plasma of 36 ng/L (i.m) and 34 ng/L (s.c) at 77 days of treatment. These levels were also significantly higher (p<0.01) by 2 to 3 fold compared to the same plasmid without a chitosan based delivery system (FIG. 7).

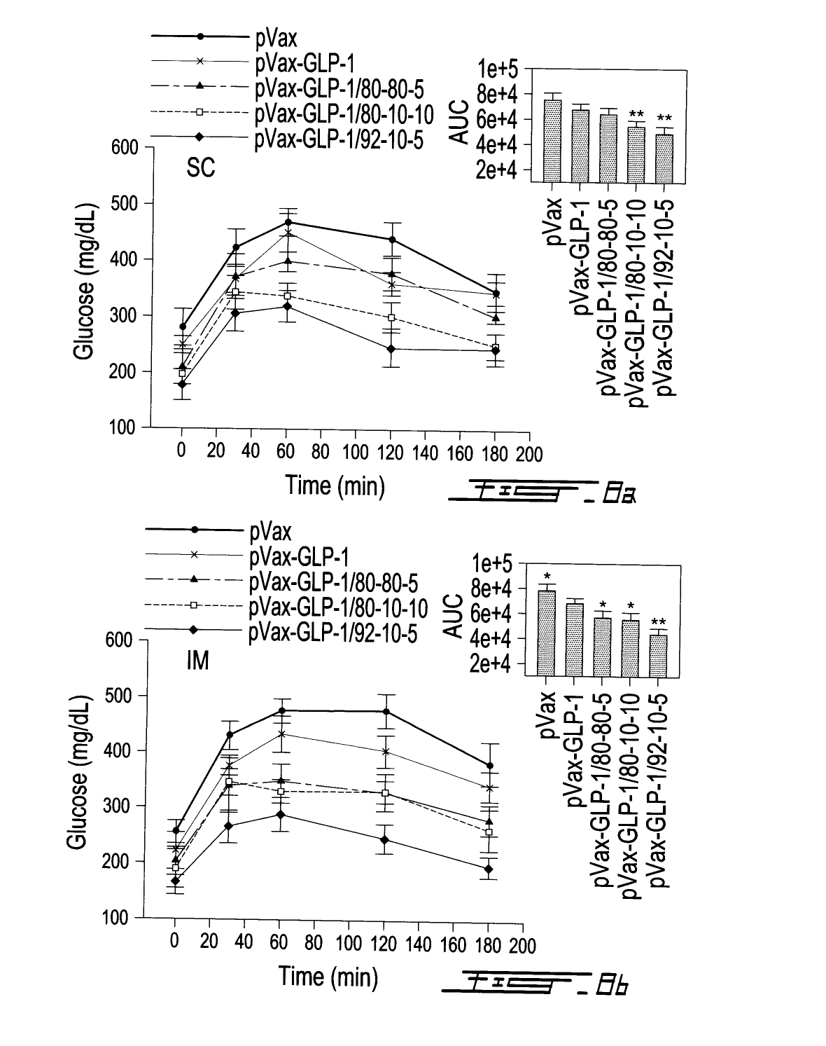

Intraperitonial glucose tolerance tests results are disclosed herein where the glucose level showed a marked decrease (better glucose tolerance, 300 mg/dL) within 2 h to reach a quasi-normal level of blood glucose at 3 h, for ZDF rats treated with intramuscular injection of nanoparticles (FIG. 8). The largest and most significant (p<0.01) decrease corresponded to the chitosan formulation 92-10-5 which also produced the highest circulating GLP-1 levels and the greatest expression in HepG2 cells in vitro (FIG. 6) thus clearly relating the therapeutic efficacy to the efficiency of this specific delivery system.

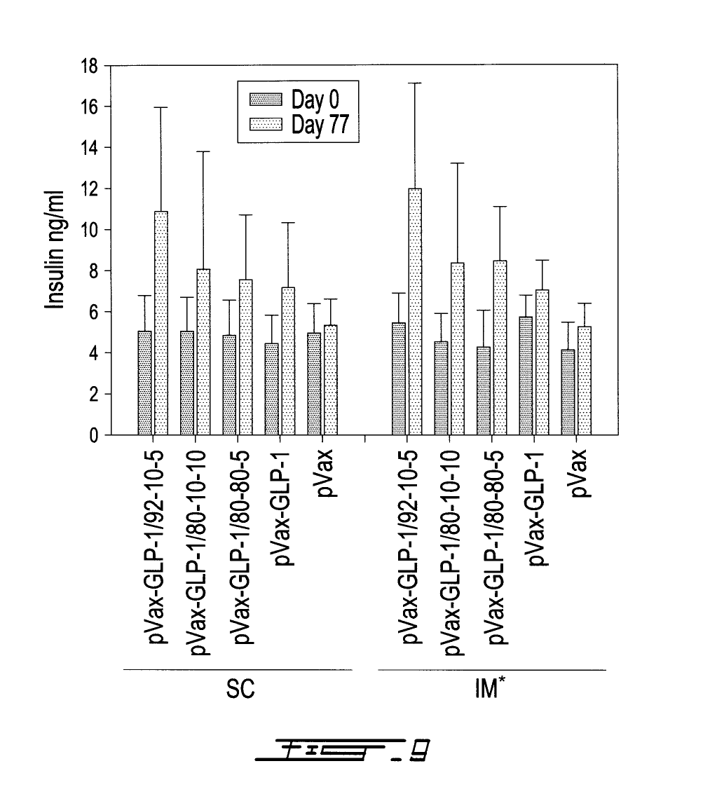

Previous work has demonstrated the effect of the GLP-1 peptide on insulin secretion and β-cells proliferation. In order to assess the effect of the chitosan based delivery of GLP-1 on insulin production, ELISA quantification of insulin in ZDF rats injected with nanoparticles either intramuscularly or subcutaneously is disclosed. Injection of pVax1 (negative control) and non treated rats did not increase insulin production (5 ng/L), whereas pVax1-GLP-1 naked show a slight increase in insulin production (7 ng/L) (FIG. 9). Insulin production following chitosan-based formulations injection was increased by two fold when compared to untreated rats. According to statistical analysis, the treatment had a significant effect on insulin levels observed on Day 77 (FIG. 9). A specific trend (p=0.08) for increased insulin expression with chitosan 92-10-5/pVax1-GLP-1 nanoparticles treated animals (12 ng/L) compared to pVax-GLP-1 without chitosan (7 ng/L) is also demonstrated. These statistical results are consistent with the higher expression level of pVax1-GLP-1 with the chitosan 92-10-5 formulation in FIGS. 6 and 7. Chitosan/pVax1-GLP-1 formulations thus permit GLP-1 expression that increases insulin production.

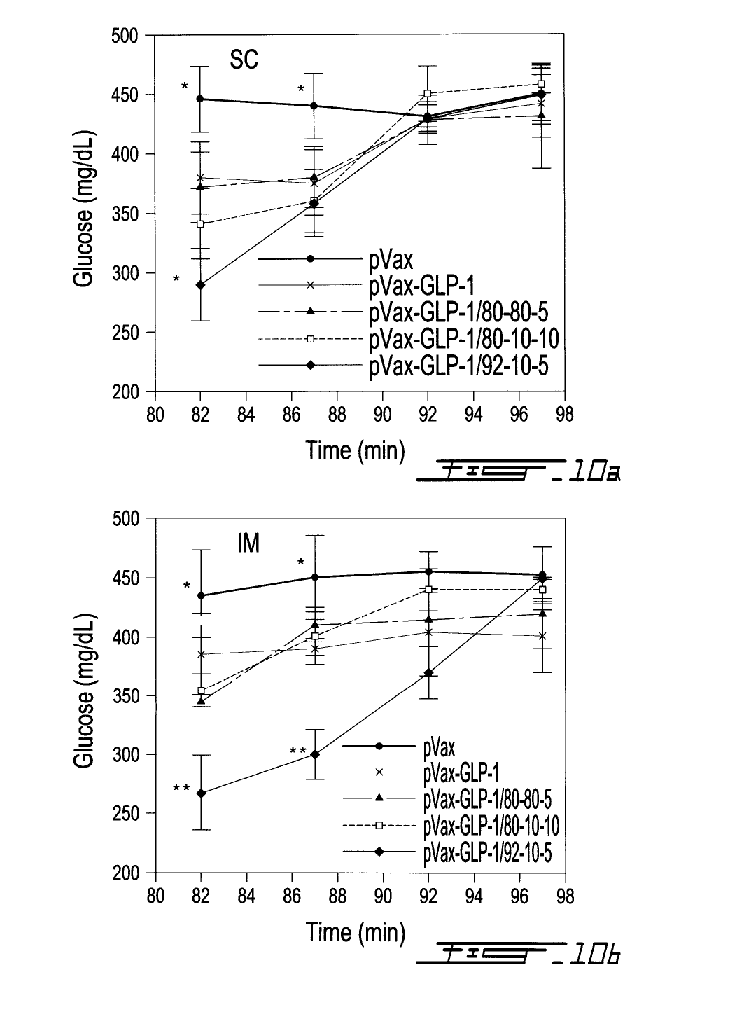

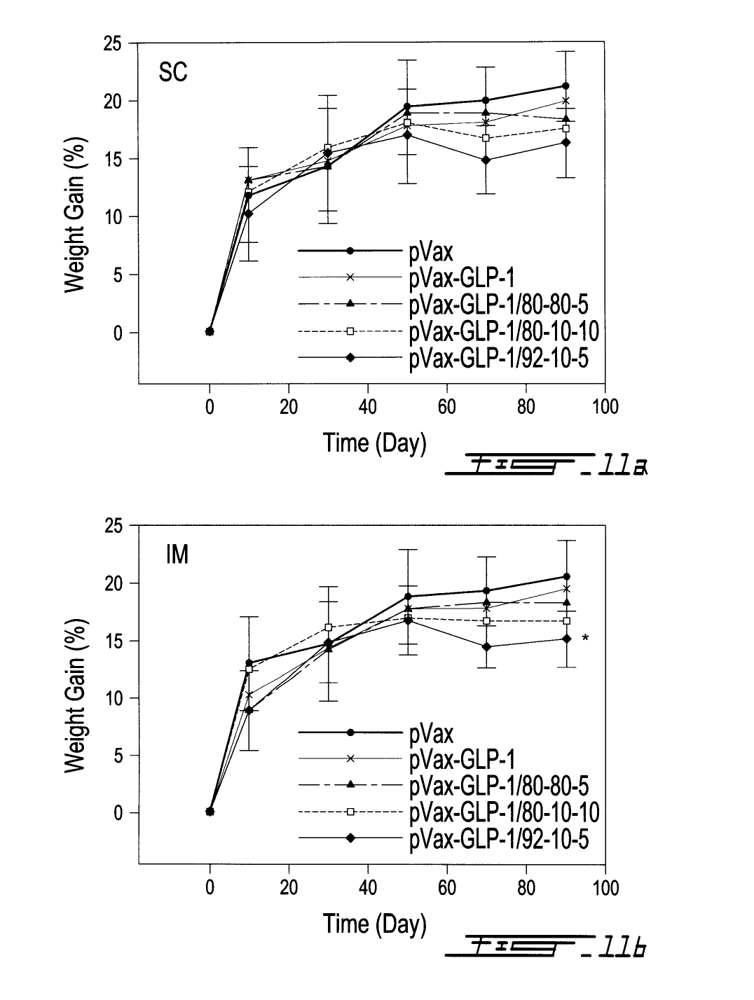

Animals treated with the nanoparticles of GLP-1 plasmid with chitosan 92-10-5 showed a decrease of blood glucose level for more than 24 days after treatment (FIG. 10), where intramuscular injections of this formulation allowed near-normalization of blood glucose level, while subcutaneous injections decreased less the blood glucose level. Other chitosan formulations allowed a sustained decrease in glucose blood level for a shorter period of time (19 days) when compared with chitosan 92-10-5. Furthermore, the weight variation in ZDF treated rats was measured in order to determine chitosan/pVax1-GLP-1 effect on weight gain during the total length of the study (90 days). Untreated and naked-pVax1 treated rats show an increase in weight of 20% during the first 50 days of the study. Their weight showed a plateau effect for 40 days. Chitosan-based formulations injected rats demonstrated a weight increase of only 15%, 5% lower than untreated at 70 days (FIG. 11).

Among the GLP-1 variants disclosed is native GLP-1(7-37), DPP-IV resistant Ser⁸-GLP-1(7-37) variant and Tyr⁹-GLP-1(7-37) variant. (FIGS. 1, 2 and 6). It has been demonstrated that the N-terminal histidine residue (His⁷) is very important for insulinotropic activity of GLP-1. For this reason the start codon was incorporated downstream from a sequence coding for Arg-Ser-Arg-Arg (SEQ ID NO:9), a signal for precursor cleavage catalyzed by furin. In mammalian cells, furin is localized to the protein secretory pathway between the trans-Golgi and cell surface. The consensus recognition sequence for furin proteases is X-Arg-X-Lys/Arg-Arg-X (SEQ ID NO:10) with the protein cleaved between the final Arg and X residues. In the construct disclosed herein, the final X is His of His⁷-GLP-1 (7-37) variant (Nakayama, 1997, Biochem J, 327: 625-635; Van de Ven et al., 1991, Enzyme, 45: 257-270).

Chitosan 92-10-5 is more efficient for the expression of recombinant GLP-1 and its variants (30-120 ng/L) when comparing to the other chitosan formulation 80-10-10 or 80-80-5 (FIG. 6). Moreover, modifications performed on the GLP-1 sequence ([Ser⁸-GLP-1(7-37)] and [Tyr⁹-GLP-1(7-37)] yield a much more resistant form of GLP-1 to DPP-IV degradation. Furthermore, modified recombinant pVax1/[Ser⁸-GLP-1 (7-37)] (>100 ng/L) or pVax1/[Tyr⁹-GLP-1 (7-37)] (>100 ng/L) show a fourfold expression increase when compared with the non modified recombinant pVax1/GLP-1 (30 ng/L) (FIG. 6).

The zeta potential of the nanoparticles diminishes with an increase of pH and to a lesser extent, with a decrease of chitosan's DDA. Furthermore, Layertu et al. (2006, Biomaterials, 27: 4815-4824) showed that molecular weight does not significantly affect the zeta potential. As reported in several studies (Ishii et al., 2001, Biochim Biophys Acta, 1514: 51-64), the zeta potential decreases when the pH rises, due to neutralization of amine groups on chitosan. The pKa of chitosan is reported to be 6.5, explaining the significant reduction in zeta potential observed when pH rises from 6.5 to 7.1. The results disclosed herein show that chitosan 92-10 formulation has a higher zeta potential (32±3.4 mV) than the chitosan 80-10 (31.1±1.3) formulation. Furthermore, the zeta potential of the formulation demonstrates their ability to bind nucleic acid (see Table 1).

The present composition can be administered with any known combination therapy, such as the co-administration of small interference RNA's (siRNAs) that can increase or decrease expression of a therapeutic protein associated with a metabolic disorder. It also encompasses any co-administration of a suitable delivery reagent such as, but not limited to, Mirus Transit TKO® lipophilic reagent, Lipofectin®, Lipofectamine™, Cellfectin®, polycations (e.g., polylysine) or liposomes.

Concurrent administration” and “concurrently administering” as used herein includes administering a composition as described herein and insulin and/or a hypoglycemic compound in admixture, such as, for example, in a pharmaceutical composition, or as separate formulation, such as, for example, separate pharmaceutical compositions administered consecutively, simultaneously, or at different times.

Suitable hypoglycemic compounds include, for example, metformin, acarbose, acetohexamide, glimepiride, tolazamide, glipizide, glyburide, tolbutamide, chlorpropamide, thiazolidinediones, alpha glucosidase inhibitors, biguanindine derivatives, and troglitazone, and a mixture thereof.

Administration of the composition described herein can be a parenteral administration which includes subcutaneous, intramuscular, intradermal, intramammary, intravenous, and other administrative methods known in the art.

The present invention will be more readily understood by referring to the following examples.

### Example I

### Preparation of Chitosan

Ultrapure chitosan samples were used where quality controlled manufacturing processes eliminate contaminants including proteins, bacterial endotoxins, toxic metals, inorganic and organic impurities. All chitosans had less than 50 EU/g of bacterial endotoxins. These chitosans were produced by heterogenous deacetylation resulting in a block rather than random distribution of acetyl groups Chitosans were selected having a 92% and 80% of degree of deacetylation and molecular weight of approximately 10 kDa and 80 kDa were produced by chemical degradation using nitrous acid as described previously (Layertu 2006).

Depolymerized chitosans were dissolved overnight on a rotary mixer at 0.5% (w/v) in hydrochloric acid using a glucosamine:HCL ratio of 1:1. Chitosan solutions were then diluted with deionized water to reach the desired amine to phosphate ratio when 100 μl of chitosan would be mixed with 100 μl of pVax-GLP-1, the latter at a concentration of 0.33 μg/μl in endotoxin-free double distilled water. Prior to mixing with pVax-GLP-1, the diluted chitosan solutions were sterile filtered with a 0.2 μm syringe filter. Chitosan/pVax-GLP-1 nanoparticules were then prepared by adding a 1:1 volume of chitosan and pVax-GLP-1 at room temperature by pipetting up and down and tapping the tube gently. The nanoparticles were incubated for 30 minutes prior to transfection.

### Example II

### Cell Line Dependencies Testing

At least two cell types in each category (DPP-IV expressing cells and DPP-IV non-expressing cells) were tested to assess cell line dependencies. HepG2, Caco2, HT-29, HEK293 and HeLa cells were cultured in MEM medium supplemented with 10% FBS. HeLa and HT29 were cultured in McCoy's and DMEM media, respectively, supplemented with 10% FBS at 37° C. and 5% CO₂. HepG2, Caco-2 and HT-29 cell line expresses dipeptidyl peptidase IV (DPP IV) and represent model cell lines in diabetes research. Cells were subcultured according to ATCC recommendations without any antibiotics. For transfection, HT-29, HepG2, HEK293, HeLa and Caco-2 cells were plated in 24-well culture plates using 500 μl/well of complete medium and 300,000 cells/well, incubated at 37° C. and 5% CO₂. The cells were transfected the next day at 50% confluency.

Complete transfection media were equilibrated overnight at 37° C. and 5% CO₂ and pH adjustment was performed with sterile HCl (1N) just before transfection. MES was added to DMEM HG and sodium bicarbonate concentration was decreased accordingly. Medium over cells was aspirated and replenished with 500 μl transfection medium containing chitosan/pVax1-GLP-1 nanoparticles at a concentration of 0.33 μg pVax1-GLP-1/well, unless otherwise noted. Cells were incubated with chitosan/pVax1-GLP-1 nanoparticles until analysis at 48 hours post-transfection. Lipofectamine™ was used as a positive control and both untreated cells and pVax1 (GLP-1 lacking plasmid) treated cells were used as negative controls.

### Example III

### DNA Plasmid Protection

The ability of the nanoparticles to protect plasmid DNA (pDNA) sequences was assessed using a DNAse protection assay. Nanoparticles of chitosan/pDNA (6 μl) were incubated in a buffer containing (pH 6.5) 20 mM MES, 1 mM MgCl₂ and a concentration of 0, 0.5, 1, 2, 5 or 10 units of DNase I. samples are incubated for 30 minutes at 37° C. The reaction was stopped by adding 2 μl of EDTA (50 mM). To ensure proper migration of the remaining pDNAs, samples were treated with Streptomyces griseus type III chitosanase at 10 mU/μL for 1.5 hours at 37° C. Samples were migrated at 90 V during 1 hour then stained with ethidium bromide (0.5 μg/mL) before visualization. The results demonstrate the ability of the formulations to protect plasmid DNA (FIG. 4). The protection is considerable and account for approximately 70% of pVac-GLP-1 when using 2 units of DNAse I/μg of DNA whereas the negative control is completely digested when 0.5 units of DNAse I per μg of DNA is used. The protection remains efficient when increasing DNAse I concentration to 5 units per μg of DNA.

### Example IV

### Assessment of Particle Uptake

Confocal microscopy was used in order to assess particle uptake and internalization into HepG-2 cell line described herein. Chitosan was labeled using rhodamine whereas pVax-1-GLP-1 plasmid was labeled using FITC. Following the labeling process, nanoparticles were formed by mixing 1:1 volume of chitosane-rhodamine and pVax1-GLP-1-FITC plasmid using the procedure described above. The formulations described were efficiently internalized into HepG-2 cells with a maximum release of pVax1-GLP-1 plasmid 24 hours post transfection. The chitosan formulation consisting of a DDA of 92% and an MW of 10 kDa showed the highest efficiency of intracellular release of pVax1-GLP-1 plasmid 24 hours post transfection. Time course studies showed that particle internalization started within an hour post transfection with a slow dynamics of endo-lysosomal sequestration and intracellular release. Intracellular release increased with time (FIG. 5) to reach a maximum near 24 hours post-transfection. These results reveal the capability of the formulation described to transfect and efficiently delivers therapeutic plasmids into different cell lines that are pertinent to treatment of Diabetes.

### Example V

### Measurement of GLP-1 Level

To assess its level, biologically active GLP-1 (7-37) concentration was determined 48 h after transfection by ELISA (Linco Research). Importantly, the ELISA system uses anti-GLP-1 antibody directed against the active form of GLP-1. Directly after GLP-1 capture, wells were washed using the washing buffer provided by the manufacturer and incubation with an alkaline phosphatase labeled anti-GLP-1 was performed. Following incubation, washing and relative fluorescence measurements (355 nm/460 nm) were performed. GLP-1 quantities (ng/L) were calculated from a standard GLP-1 curve (FIG. 5).

### Example VI

### Assessment of GLP-1 Gene Therapy

For in vivo assessment of GLP-1 gene therapy, the Zucker Diabetic Fatty (ZDF) rat was chosen, a model which spontaneously develops type 2 diabetes (non-insulin dependent diabetes mellitus or NIDDM) (Brunner et al., 2000, Gene Ther, 7: 401-407). Nanoparticles were administrated to ZDF rats via either intra muscular (i.m) or subcutaneous (s.c) injection (100 μg DNA) at each of days 0, 7, 14, 21, 35, 49 and 63. In vivo expression of GLP-1 was assessed using the ELISA (Linco Research) on nanoparticle treated ZDF rat plasma samples. Prior to ELISA quantification of active GLP-1, blood samples were centrifuged for 10 min at 1000×g in order to recover plasma (FIG. 6).

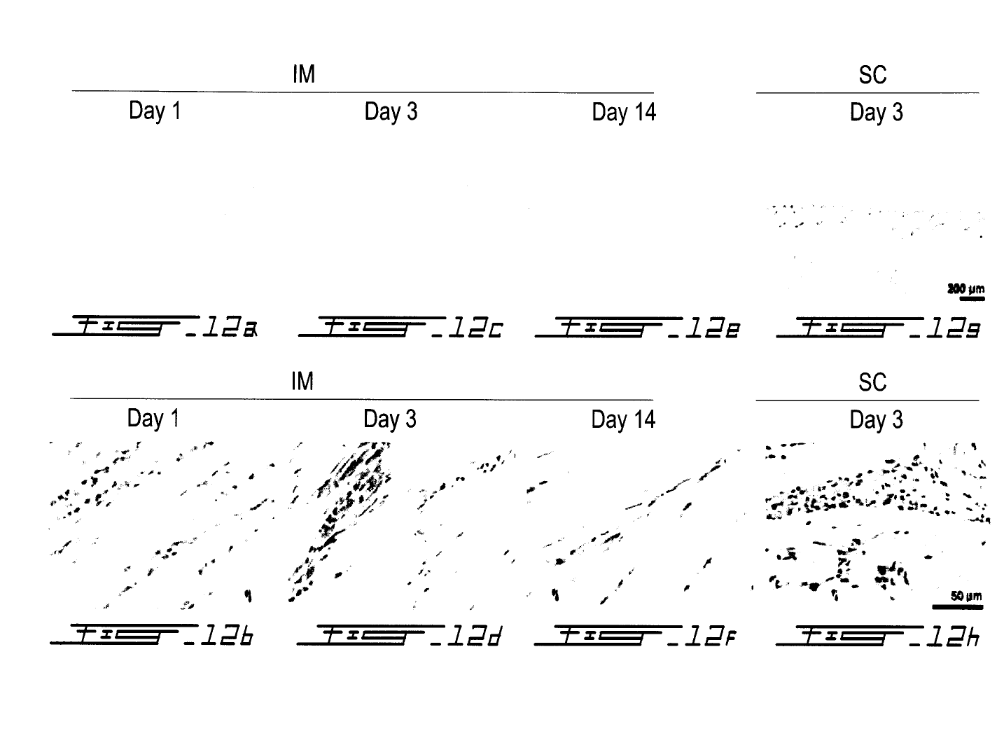

Histological analysis of treated ZDF rats was performed on skin and muscle tissues derived from the injection sites. Tissues were dehydrated using alcohol and paraffin embedded. Tissue sectioning (4-6 μm) was performed using the Leica™ RM 2155 microtome (Leica™ Microsystems, Deerfield, Ill.). Prior to Safranin-O (1%)/Fast-Green (0.04% w/v)/iron Haematoxylin staining, tissue sections were deparaffinised and rehydrated. Images were taken using the Zeiss Axiolab microscope combined to an analogue Hitachi NV-F22 camera (FIG. 12).

### Example VII

### Assessment of Size of Chitosan/pVax1-GLP-1 Nanoparticles

Size of chitosan/pVax1-GLP-1 nanoparticles was determined by dynamic light scattering at an angle of 137° at 25° C. using a Malvern Zetasizer Nano ZS (Table 1). Samples were measured in triplicates using refractive index and viscosity of pure water in calculations. The zeta potential was measured in triplicates as well using laser Doppler velocimetry at 25° C. using the same instrument and the dielectric constant of water for calculation. For the size determination, 200 μl of chitosan was mixed with 200 μl of pVax-GLP-1) then completed to 500 μl using 10 mM NaCl. For zeta measurement, nanoparticles were diluted 1:2 using 500 μl of 10 mM NaCl (Table 1).

TABLE 1
Size, zeta potential, pH and osmolarity values
for chitosan/pVax-GLP-1 nanoparticles

| | size | Zeta poten- | | |
|---|---|---|---|---|
| Sample | (nm) | tial (mV) | pH | mOsm |
| Chitosan 92-10-5/pVax-GLP-1 | 235 ± 48 | 32.0 ± 3.4 | 4.8 | 22 |
| Chitosan 80-10-10/pVax-GLP-1 | 163 ± 22 | 26.7 ± 3.9 | 3.7 | 30 |
| Chitosan 80-80-5/pVax-GLP-1 | 246 ± 30 | 31.1 ± 1.3 | 4.8 | 20 |

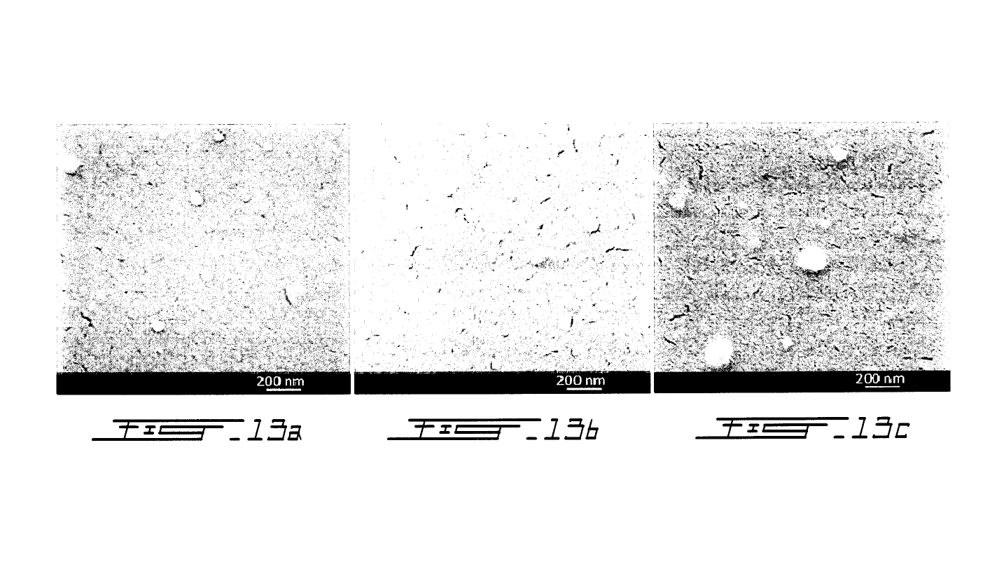

Nanoparticle size and form were also assessed using an environmental electron scanning microscope (ESEM). Chitosan/pVax-GLP-1 nanoparticles were vaporized on a silica surface of 1 cm² then coated with gold using the Agar sputter coater machine (MARIVAC Inc). Samples were scanned using the high vacuum mode. The results show nanoparticles of predominantly spherical shape ranging between 150-250 nm depending on the size of the plasmid and of the chitosan used (FIG. 13). Smaller plasmids generate smaller particles, probably since the plasmid forms the structural core of the nanoparticles, while longer chitosan chains produce smaller particles, due to their greater DNA condensing capacity. Results obtained with specific formulations described herein are consistent with dynamic light scattering results obtained before. Furthermore, the method described herein yields reproducible size results allowing the bypass of renal clearance thus improving in vivo transfection efficiency and increasing circulating nanoparticle half life.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A composition comprising chitosan and a plasmid DNA sequence encoding for Glucagon like peptide-1 (GLP-1), a GLP-1 variant or a GLP-1 derivative.

2. The composition of claim 1, wherein the GLP-1 variant is GLP-1(7-34), GLP-1(7-35), GLP-1(7-36), Val⁸-GLP-1(7-37), Gln⁸-GLP-1(7-37), D-Gln⁹-GLP-1(7-37), Thr¹⁸-Lys¹⁸-GLP-1(7-37), Lys¹⁸-GLP-1(7-37), His⁷-GLP-1 (7-37), Ser⁸-GLP-1(7-37) or Tyr⁸-GLP-1(7-37).

3. The composition of claim 2, wherein the GLP-1 variant is SEQ ID NO:3 or SEQ ID NO:4.

4. The composition of claim 1, wherein the chitosan is heterogeneously deacetylated.

5. (canceled)

6. The composition of claim 1, wherein the plasmid DNA comprises an expression facilitating sequence derived from a CMV promoter (CMV Pro); a sequence coding for a furin cleavage site (FCS); and a sequence coding for GLP-1, GLP-1 variant or GLP-1 derivative thereof that is operably linked to said expression facilitating sequence.

7. The composition of claim 1, wherein the plasmid DNA is pVax1 plasmid.

8. The composition of claim 1, wherein the chitosan has a molecular weight of 5 kDa to 150 kDa and a deacetylation degree (DDA) of 75% to 95%.

9. The composition of claim 8, wherein the molecular weight of the chitosan is 5 to 15 kDa and the DDA is 90% to 95%.

10. The composition of claim 1, wherein the ratio of amine groups on chitosan to phosphate groups of plasmid DNA (N:P ratio) is in the range of 2 to 20.

11. The composition of claim 10, wherein the N:P ratio is of 3 to 10.

12. The composition of claim 1, wherein the chitosan comprises block distribution of acetyl groups or a chemical modification.

13-41. (canceled)

42. A method for treating diabetes mellitus or related conditions, controlling glucose metabolism, or for treating a metabolic disease in a patient comprising administering an effective amount of the composition as defined in claim 1.

43. The method of claim 42, wherein said diabetes mellitus related conditions are insulin-dependent diabetes mellitus (type I diabetes), noninsulin-dependent diabetes mellitus (type II diabetes), insulin resistance, hyperinsulinemia, diabetes-induced hypertension, obesity, damage to blood vessels, damage to eyes, damage to kidneys, damage to nerves, damage to autonomic nervous system, damage to skin, damage to connective tissue, and damage to immune system.

44. (canceled)

45. The method of claim 42, wherein said composition reduces the blood glucose level in said patient.

46. (canceled)

47. The method of claim 42, wherein said composition reduces the weight gain in said patient.

48. The method of claim 42, further reducing circulating half life of incretins, incretin-like proteins, or glycoregulating proteins.

49. The method of claim 42, further increasing insulin secretion and β-cells proliferation.

50. The method of claim 42, wherein the composition is administered by a subcutaneous administration, an intramuscular administration, an intravenous administration, an intradermal administration, intramammary administration, an intraperitoneal administration, an oral administration or a gastrointestinal administration.

51. The method of claim 42, wherein the composition further comprises insulin or a hypoglycemic compound.

52. The method of claim 42, further comprising administering a small interference RNA's (siRNAs), a suitable delivery reagent, insulin or a hypoglycemic compound.

53.-54. (canceled)


SEQUENCE LISTING (10 sequences)

SEQ ID NO: 1 - DNA, 1076 bp, Artificial Sequence
  Proglucagon encoding gene
agaagggcagagcttgggcgcagaacacactcaaagttcccaaaggagctccacctgtctacacctcctctcagctcagtcccacaaggcagaataaaaaatgaagaccgtttacatcgtggctggattgtttgtaatgctggtacaaggcagctggcagcatgcccctcaagacacggaggagaacgccagatcattcccagcttcccagacagaaccacttgaagaccctgatcagataaacgaagacaaacgccattcacagggcacattcaccagtgactacagcaaatacctagactcccgccgtgctcaagattttgtgcagtggttgatgaacaccaagaggaaccggaacaacattgccaaacgtcatgatgaatttgagaggcatgctgaagggacctttaccagtgatgtgagttcttacttggagggccaggcagcaaaggaattcattgcttggctggtgaaaggccgaggaaggcgagacttcccggaagaagtcgccatagctgaggaacttgggcgcagacatgctgatggatccttctctgatgagatgaacacgattctcgataaccttgccaccagagacttcatcaactggctgattcaaaccaagatcactgacaagaaataggaatatttcaccattcacaaccatcttcacaacatctcctgccagtcacttgggatgtacatttgagagcatataccgaagctatactgcttggcatgcggacgaatacatttccctttagcgttgtgtaacccaaaggttgtaaatggaataaagtttttccagggtgttgataaagtaacaactttacagtatgaaaatgctggattctcaaattgtctcctcgttttgaagttaccgccctgagattacttttctgtggtataaattgtaaattatcgcagtcacgacacctggattacaacaacagaagacatggtaacctggtaaccgtagtggtgaacctggaaagagaacttcttccttgaaccctttgtcataaatgcgctcagctttcaatgtatcaagaatagatttaaataaatatctcatc

SEQ ID NO: 2 - DNA, 96 bp, Artificial Sequence
  GLP-1 (7-37)
catgctgaagggacctttaccagtgatgtgagttcttacttggagggccaggcagcaaaggaattcattgcttggctggtgaaaggccgaggatag

SEQ ID NO: 3 - DNA, 96 bp, Artificial Sequence
  Ser8-GLP-1 (7-37)
cattctgaagggacctttaccagtgatgtgagttcttacttggagggccaggcagcaaaggaattcattgcttggctggtgaaaggccgaggatag

SEQ ID NO: 4 - DNA, 96 bp, Artificial Sequence
  Tyr9-GLP-1 (7-37)
catgcttatgggacctttaccagtgatgtgagttcttacttggagggccaggcagcaaaggaattcattgcttggctggtgaaaggccgaggatag

SEQ ID NO: 5 - DNA, 14 bp, Artificial Sequence
  RV-GLP-1(7-37)
tcctcggcctttct

SEQ ID NO: 6 - DNA, 15 bp, Artificial Sequence
  FW-GLP-1(7-37)
catgctcaagggacc

SEQ ID NO: 7 - DNA, 15 bp, Artificial Sequence
  FW-[Ser8]GLP-1(7-37)
cattctcaagggacc

SEQ ID NO: 8 - DNA, 15 bp, Artificial Sequence
  FW-[Tyr9]GLP-1(7-37)
catgcttatgggacc

SEQ ID NO: 9 - protein, 4 aa, Artificial Sequence
  Signal for precursor cleavage catalyzed by furin
RSRR

SEQ ID NO: 10 - protein, 6 aa, Artificial Sequence
  Consensus recognition sequence for furin
XRXXRX